(19) United States
(12) Patent Application Publication
(10) Pub. No.: US 2023/0134706 A1
(43) Pub. Date: May 4, 2023

(54) SENSORED COMPONENT FOR ENDOSSEOUS SCREW, SCREW ASSEMBLY COMPRISING SAID COMPONENT, AND MONITORING SYSTEM COMPRISING SAID COMPONENT

(71) Applicant: Orthofix S.r.l., Bussolengo (VR) (IT)

(72) Inventors: Mario Donnici, Bussolengo (VR) (IT); Federico Vicenzi, Verona (VR) (IT); Daniele Venturini, Povegliano Veronese (VR) (IT); Gaetano Marrocco, Monte Compatri (RM) (IT); Cecilia Occhiuzzi, Roma (IT); Sara Amendola, Roma (IT); Simone Nappi, Latina (IT); Carolina Miozzi, Roma (IT)

(21) Appl. No.: 17/974,324

(22) Filed: Oct. 26, 2022

(30) Foreign Application Priority Data
Oct. 28, 2021 (IT) 102021000027635

(51) Int. Cl. A61C 8/00; H01Q 1/22

(52) U.S. Cl. CPC A61C 8/0093; H01Q 1/22

(57) ABSTRACT

Component (1) for endosseous screw, provided with a main body (10) which supports electronics (2) comprising at least one signal emission circuit and at least one sensor for acquiring one or more biophysical parameters of a patient, said component (1) being associable with an endosseous screw.

### PRIORITY

This application claims priority to and the benefit of the filing date of Italian Patent Application No. 102021000027635 filed Oct. 2, 2021, incorporated herein by reference in its entirety.

### TECHNICAL FIELD

The present invention relates to a sensored component for the application on an endosseous screw, to a screw assembly comprising said sensored component, and to a monitoring system comprising said sensored component.

Therefore, the invention finds useful application in the field of orthopedics, in particular corrective orthopedics.

More specifically, the invention may find useful application in treating structural collapses and joint bone deformations due to the Charcot neuro-osteoarthropathy, for instance in treating the so-called Charcot foot, and the following description is made with non-limiting reference to its use in the context of said application.

### BACKGROUND

Various orthopedic techniques provide for the application, temporarily or permanently, of devices, implants, or grafts which, in order to attach to the patient's bone sites, are totally or partially inserted into the body thereof.

The introduction of foreign bodies into the patient's body requires monitoring the situation over time, both to verify the correct positioning and load of the structures, and above all to early diagnose any infections.

If up to now we have mainly resorted to outer observations for the aforementioned monitoring, mostly relying on physical or radiological semiotics, in recent times the possibility of integrating sensors into implanted devices has been investigated, in order to acquire significant parameters such as the loads on the structure, temperature, movement, pH, presence of enzymes or bacteria, and more. The data acquired by the internal sensors may be used both in real time in the intra-operative period, to assist the surgeon during the implantation of the device, and in subsequent moments for the actual post-operative monitoring. In this second perspective, the data may be acquired upon request, by querying the sensored system at regular intervals by means of an outer reading device, or they may trigger an alarm on a reading device only when certain thresholds are reached. It is even possible to contemplate a hypothesis of automatic administration of a drug indicated by the implant itself upon reaching specific alarm thresholds for the parameters monitored by the sensors.

Despite the potential advantages mentioned above, the integration of sensors in the various internal orthopedic implants involves substantial difficulties. In particular, it should be considered that implants are often the result of careful product development and a long clinical trial phase. The integration of sensors and of the corresponding electronics for the acquisition of signals, as well as an antenna intended for sending data to the outside, requires a complete and non-trivial redesign of the orthopedic devices currently on the market.

Among the various orthopedic devices that could benefit from the integration of sensors, endosseous screws must certainly be included, which are used for instance in treating bone failure or deformation as in the case of the so-called Charcot foot. In treatments of this type, a plurality of bone screws, generally cannulated, are introduced to recreate and support the foot arches. There is a considerable risk of infections here, for which it is vital to make an early diagnosis.

On the other hand, the bone screw has a structure uniquely designed for its function, and the modification of its structure for housing the electronics and sensors would cause mechanical-structural consequences that would make new mechanical and medical characterizations and certifications necessary for marketing the modified device.

The technical problem underlying the present invention is to provide a device that allows the integration of a sensor on an orthopedic implant, in particular on an endosseous screw, without requiring a complete redesign of the implant itself

### SUMMARY OF THE INVENTION

The solution idea underlying the present invention is to propose a sensored component which associates with the endosseous screw, without modifying the structural mechanical properties thereof in any way.

Therefore, the above technical problem is solved by a component for endosseous screw, provided with a main body which supports electronics comprising at least one signal emission circuit and at least one sensor for acquiring one or more biophysical parameters of a patient, said component being associable with an endosseous screw, said signal emission circuit being arranged to send at least one signal relating to the one or more biophysical parameters acquired by the at least one sensor.

The sensored component may be integrally associated with the bone screw or, preferably, made as a separated piece and then coupled to the screw, in the pre-operative or intra-operative phase. Preferably, the sensored component is mechanically and removably coupled to the bone screw, for instance through threading.

The sensored component may also be adapted to endosseous screws of different diameter, length, and conformation; for instance, it is possible to make a set of components adapted to the different sizes of a fixation system comprising a plurality of screws.

Preferably, the signal emission circuit is an RFID transponder circuit. Said construction method is particularly advantageous since the sensored component does not require an internal power supply battery, which would limit the useful life of the implant. Preferably, the RFID transponder works in UHF band (868-950 MHz).

The main body preferably comprises a distal portion and a proximal portion, both conductive, separated by a dielectric intermediate portion, said electronics being electrically connected to both said distal portion and to said proximal portion.

In other words, the electronics with a signal emission circuit is placed on a conductor bridge that connects said distal portion to said proximal portion. The resulting architecture defines an antenna exciter, which also uses the body of the endosseous screw as a radiating element, and wherein the gap between the two conductive portions (i.e. the thickness of the dielectric portion) is a parameter that affects the transmission.

The above thickness may have an optimal value less than 6 mm: in particular, it was noted that a value of 4 mm achieves a good antenna gain and at the same time is easily achievable with the architecture proposed herein below.

It is noted that the dielectric portion of the main body may be made of different biocompatible materials (PVC, PMMA, PEEK); experiments carried out by the Applicant show how the different dielectric constant of these materials does not significantly affect the antenna gain.

The electronics may comprise at least one integrated circuit arranged on the conductor bridge.

Said integrated circuit may comprise both the signal emission circuit and the at least one sensor therein. Moreover, inside the integrated circuit a proper adaptation circuit may be provided, which allows tuning the same component to the different RFID UHF working frequencies and to the patient's different morphologies.

Obviously, the above identified different circuits may also be provided separated and not grouped on a single integrated circuit.

The electronics may still comprise an antenna tuning inductor arranged in series in the integrated circuit on the conductor bridge.

Preferably, the integrated circuit and the conductor bridge, as well as any additional electronic components such as the antenna tuning inductor, are inserted in a side recess of the dielectric intermediate portion. The above elements for instance may be supported by a plate or slide that is press-fitted in the side recess.

The main body is preferably cylindrical, the three distal, proximal, and intermediate components being made of cylinders of equal diameter.

In a first variant, the main body is monolithic and made of a dielectric material, the distal and proximal portions being made conductive by a metallization or metal deposition process on the surface of said dielectric material.

In an alternative variant, said main body is composed by said distal and proximal portions of a conductive material being assembled at the two opposite ends of the intermediate portion of a dielectric material. The three pieces are preferably press-fitted together.

Preferably, the component according to the invention comprises an attachment portion for coupling with an endosseous screw, said attachment portion extending in a proximal position with respect to the main body. Said attachment portion, having a smaller diameter than the main body, is preferably made integrally with the conductive proximal portion of the main body, so that there is a direct electrical connection with the metal body of the endosseous screw.

Preferably, said attachment portion comprises a cylindrical stem provided with an outer thread arranged to couple with the endosseous screw. The component may thus be assembled on the implant in an extremely easy manner, by screwing into the system. Moreover, the component may comprise a coupling head for a screwing tool, for instance a hexagonal recess for the insertion of a socket head screw or two or more smooth side holes that are parallel to the longitudinal axis of the component but away therefrom. In this way, the assembly may be made directly by the surgeon.

The at least one sensor may acquire a temperature and/or a local pH of the patient, preferably both. In fact, the medical literature has shown that a variation of these parameters is a reliable indicator of infections.

The previously identified technical problem is also solved by a screw assembly comprising an endosseous screw, in turn comprising a tip, a stem, and a head, and at least one component of the previously described type coupled to the screw head.

Preferably, the endosseous screw is at least partially made of a conductive material and defines, together with the component, an antenna for the signal emission circuit. Obviously, this implies a conductive coupling between sensored component and endosseous screw, made for instance of a metal-to-metal threaded coupling.

In other words, the signal emission circuit uses both the structure of the implant and the stem of the endosseous screw as radiating elements to allow communicating the data relating to the prosthesis to an outer reader. From this point of view the sensored component may be considered an energy coupler from the circuit to the endosseous screw.

The Applicant noted how the antenna gain thus defined is weakly influenced by the length of the endosseous screw, at least for lengths between 80 mm and 150 mm typically used in the treatment of the Charcot foot. For this reason, it is not necessary to redesign the sensored component for the different screw lengths generally provided in a set of endosseous screws.

As above mentioned, the endosseous screw used in connection with the sensored component is arranged for the treatment of collapses and bone deformations, such as the Charcot foot, said tip and said head being both threaded, said stem being unthreaded. Still preferably, said endosseous screw is cannulated.

The Applicant has experimentally verified that the presence of other endosseous screws alongside the sensored screw, as occurs in the screw systems used in the above-mentioned reconstruction techniques, while worsening the antenna gain, does not affect the correct communication of the sensored component with an outer receiving reader.

The above identified technical problem is also solved by a monitoring system comprising a screw assembly and at least one receiving reader arranged to acquire a signal coming from the signal emission circuit defined by the screw assembly, said signal comprising at least data relating to the one or more biophysical parameters acquired by the at least one sensor.

Preferably, the receiving reader is an RFID reader.

The receiving reader may be of the handheld type, preferably wearable by the user, for instance by means of a wrist strap. Said solution advantageously allows a patient self-reading mode.

Alternatively, it is possible to provide a fixed receiving reader, that it is used for instance during periodic post-operative monitoring visits.

The receiving reader is preferably able to send a signal to a mobile device, whereon a suitable interface is installed, which allows acquiring and viewing the data sent. Alternatively, the reader may communicate with a computer or still be provided with an own reading interface.

Sending signals from the receiving reader to the mobile device and/or to the computer may occur with any available technology, even of the wireless type: for instance, through Bluetooth technology.

The receiving reader may be able to acquire and possibly forward one or more of the following data to the mobile device:
a unique identification of the user;
data relating to the endosseous screw (model, size, implant date and position);
a degree of strength of the coupling between the receiving reader and the endosseous screw;
temperature and/or local pHs in real time.

Moreover, the receiving reader may store data and send alarms should warning thresholds for temperature and pH be exceeded.

Further features and advantages will become more apparent from the following detailed description of some preferred, but not exclusive, embodiments of the present invention, with reference to the enclosed figures given by way of non-limiting example.

### BRIEF DESCRIPTION OF THE DRAWINGS

FIG. 1 represents a perspective view of a first embodiment of a sensored component according to the present invention;

FIG. 2 represents a side view sectioned along a transversal plane of the component in FIG. 1;

FIG. 3 represents a perspective view of a second embodiment of a sensored component according to the present invention;

FIG. 4 represents a side view of the component of FIG. 3;

FIG. 5 represents a side view sectioned along a transversal plane of the component of FIG. 3;

FIG. 6 represents an exploded side view of the component of FIG. 3;

FIG. 7 represents an exploded perspective view of the component of FIG. 3;

FIG. 8 shows a perspective view of an endosseous screw according to the present invention, provided with the component of FIG. 1, comprised in a fixation system and implanted on a patient's bone site;

FIG. 9 shows an enlarged detail of FIG. 8;

FIG. 10 represents a perspective view of a third embodiment of a sensored component according to the present invention;

FIG. 11 represents a side view sectioned along a transversal plane of the component in FIG. 10;

FIG. 12 represents a perspective view of a fourth embodiment of a sensored component according to the present invention;

FIG. 13 represents a side view sectioned along a transversal plane of the component of FIG. 12;

FIG. 14 represents an exploded perspective view of the component of FIG. 12;

FIG. 15 shows a perspective view of an endosseous screw according to the present invention, provided with the component of FIG. 10, comprised in a fixation system and implanted on a patient's bone site;

FIG. 16 shows an enlarged detail of FIG. 15;

FIG. 17 schematically represents a monitoring system according to the present invention.

### DETAILED DESCRIPTION

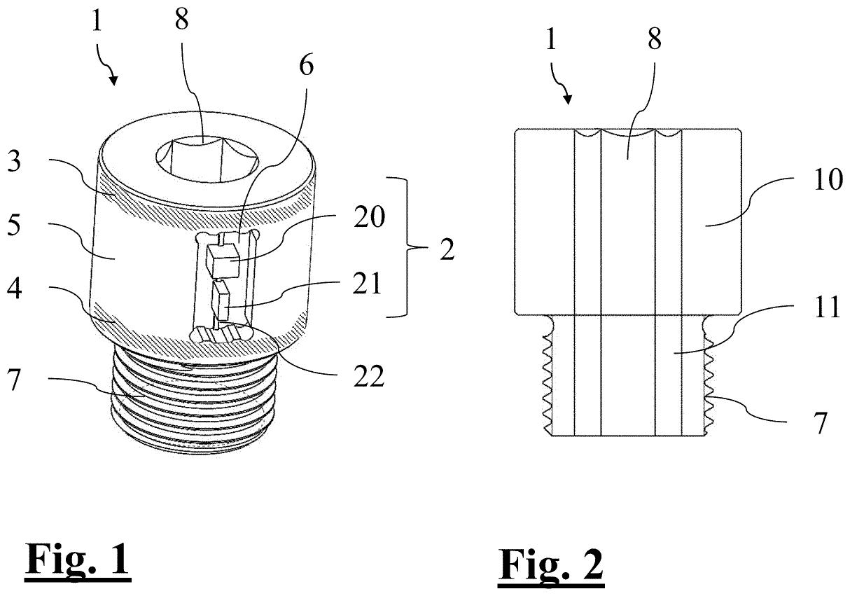

With reference to the enclosed FIGS. 1 and 2, reference number 1 identifies a first embodiment of a sensored component according to the present invention.

The component 1 comprises a cylindrical-shaped main body 10 from which an attachment portion 11 provided with an outer thread 7 extends.

The component 1 is crossed by a central hexagonal recess 8 which allows inserting a screwing tool, in particular an Allen wrench.

The main body 10 is made of a dielectric material; two opposite distal 3 and proximal 4 portions are however made conductive by a metallization or metal deposition process on the outer surface. Instead, an intermediate portion 5 is kept insulating. The metallization of the proximal portion 4 extends up to the outer thread 7.

The thickness of the intermediate portion 5, given by way of non-limiting example, is 4 mm.

The dielectric material may be PVC, PMMA, PEEK, or still another material.

The electronics 2 is arranged in a recess 6 formed at the intermediate portion 5, said electronics 2 being made of an integrated circuit 20 and of an antenna tuning inductor 21 arranged in series on a conductor bridge 22 that joins the distal portion 3 to the proximal portion 4.

The integrated circuit 20 comprises therein an RFID transponder circuit and an adaptation circuit that allows tuning the component itself to the different RFID UHF working frequencies and to the different morphologies of the patient. The RFID transponder circuit defines an RFID transponder together with the antenna, defined by the component 1 coupled to an endosseous screw 100.

The integrated circuit 20 still comprises therein sensors for measuring the patient's temperature T and pH P at the endosseous screw 100. Said measures are sent through the RFID transponder to an RFID reader 200, further described in greater detail hereinafter, in order to carry out an early diagnosis of infections due to the implantation of the endosseous screw 100.

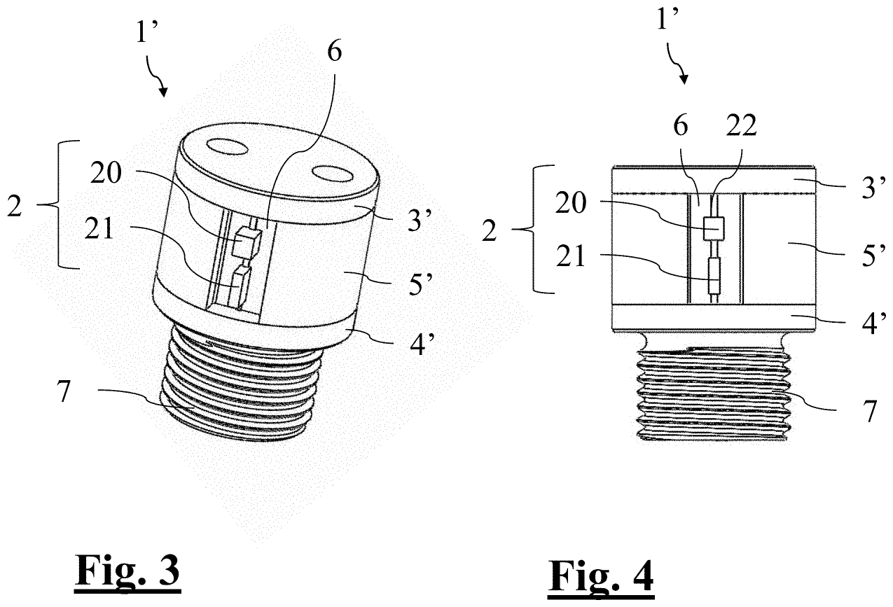

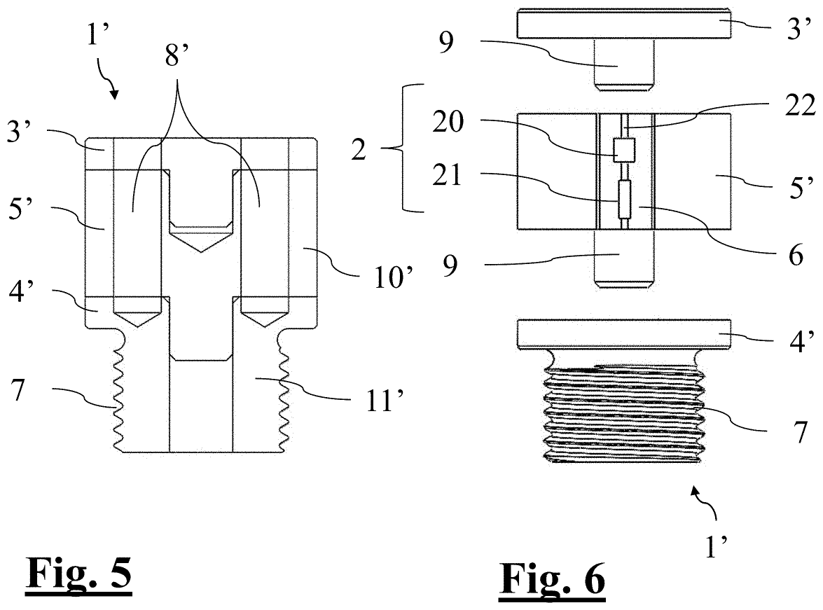

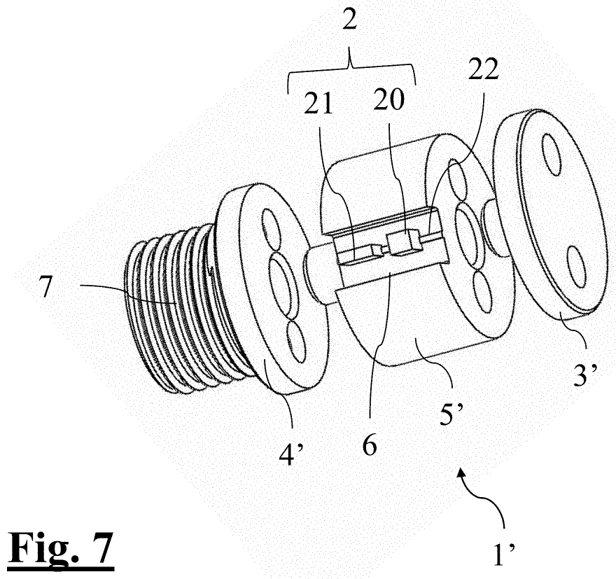

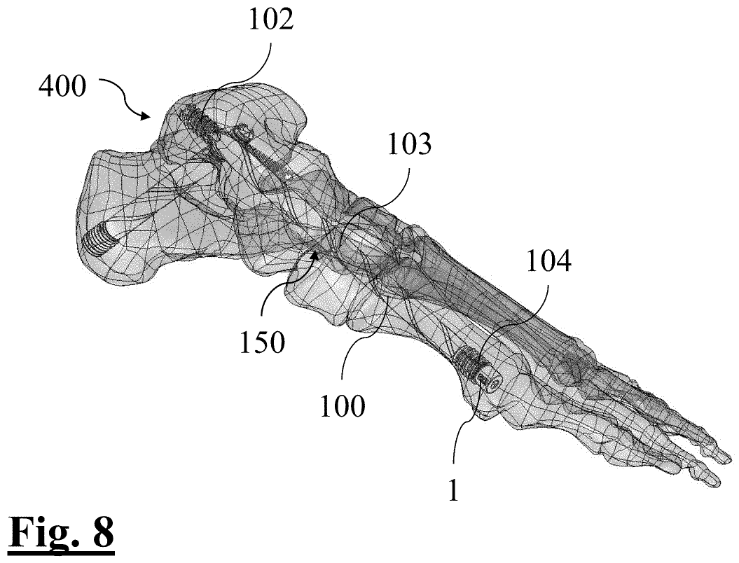

With reference to the enclosed FIGS. 3-7, reference number 1′ identifies a second embodiment of a sensored component according to the present invention.

The second embodiment differs from the first one in that the main body 10′ is not monolithic, but made of a distal portion 3′, an intermediate portion 5′ and a proximal portion 4′, which are distinct and snap fitted together. In particular, the assembling occurs through forced coupling of two pins 9 integral with the distal portion 3′ and the intermediate portion 5′ with as many holes formed in the intermediate portion 5′ and in the proximal portion 4′.

The distal portion 3′ and the proximal portion 4′ are integrally made of a conductive material, whereas the intermediate portion 5′ is made of a dielectric material. The attachment portion 11′ is made integral with the distal portion 4′, namely also made of a conductive material.

The second embodiment differs from the first one also in that the central hexagonal recess 8 is replaced by two smooth holes 8′, which are axial and parallel but away from the axis of the main body 10′. The smooth holes 8′ are used, in analogy to the hexagonal recess, for inserting a two-tipped screwing tool.

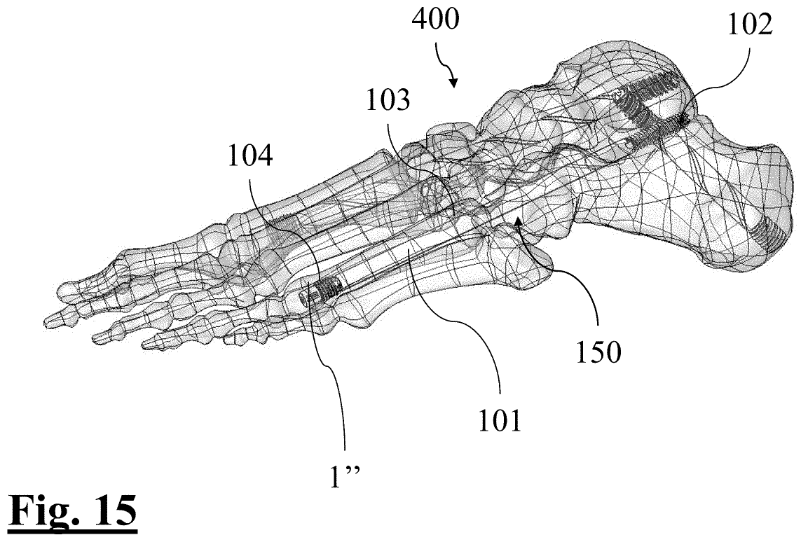

FIGS. 8 and 15 show an endosseous screw system 400 implanted on an anatomical site of the patient for the plantar arch reconstruction of a patient. Each of the bone screws 100, 101 has a threaded tip 102, a non-threaded stem 103 and a head 104, which is also threaded.

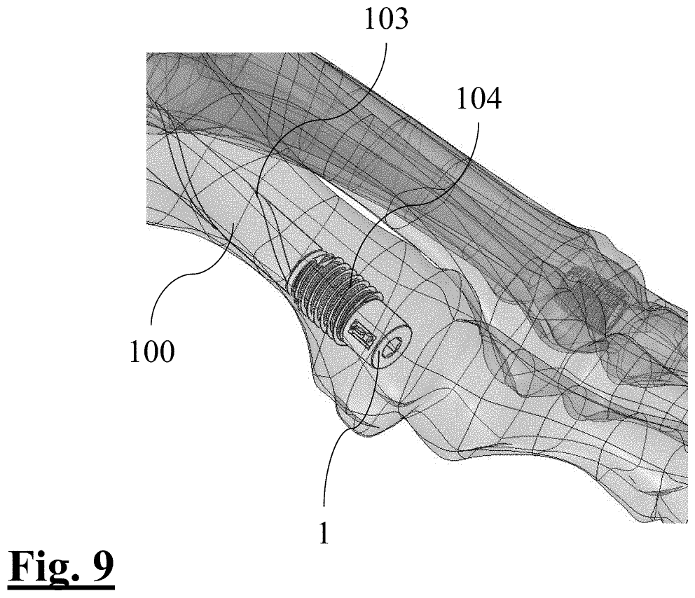

As visible in FIG. 9, the component 1 is screwed into the head 104 of one of the endosseous screws 100 of larger diameter.

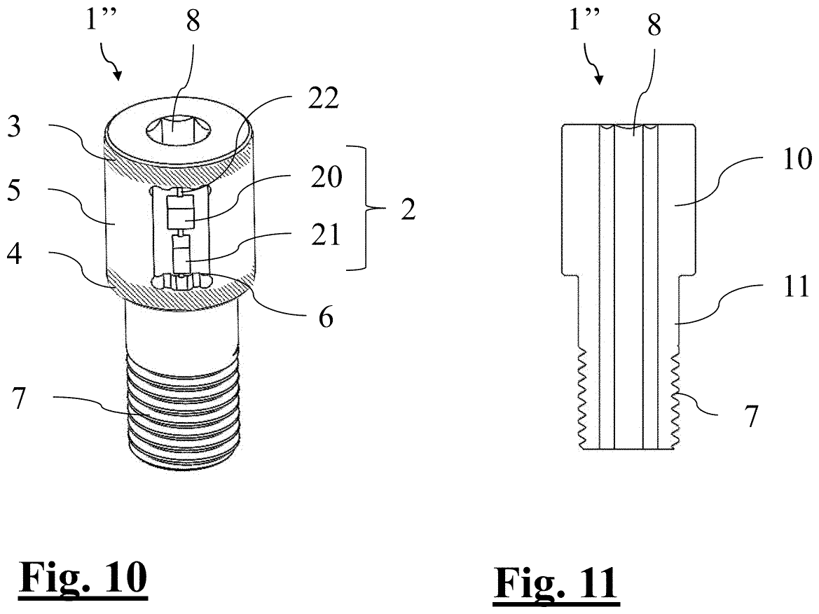

With reference to the enclosed FIGS. 10 and 11, reference number 1″ identified a third embodiment of a sensored component according to the present invention.

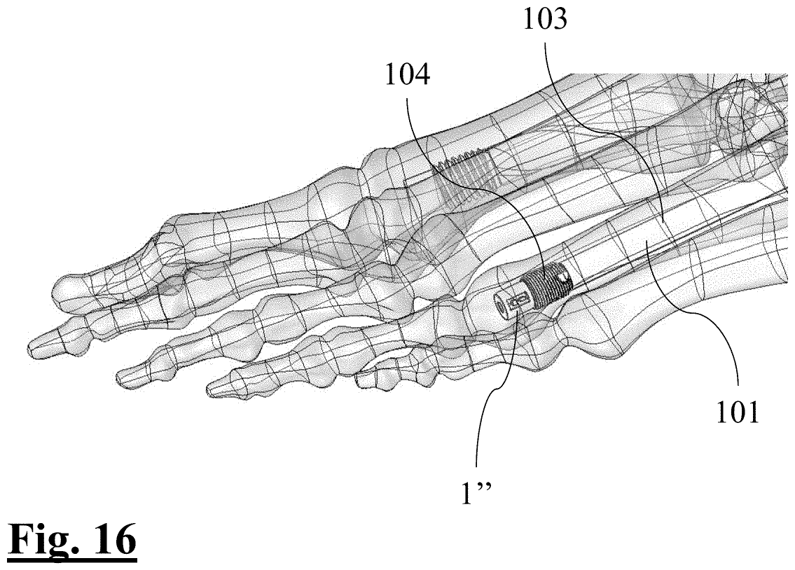

The third embodiment is substantially identical to the first one, except that, as visible in FIG. 16, it has a smaller diameter and as a result it is arranged to couple to one of the endosseous screws 101 of smaller diameter of the fixing system 400 made of a plurality of bone screws.

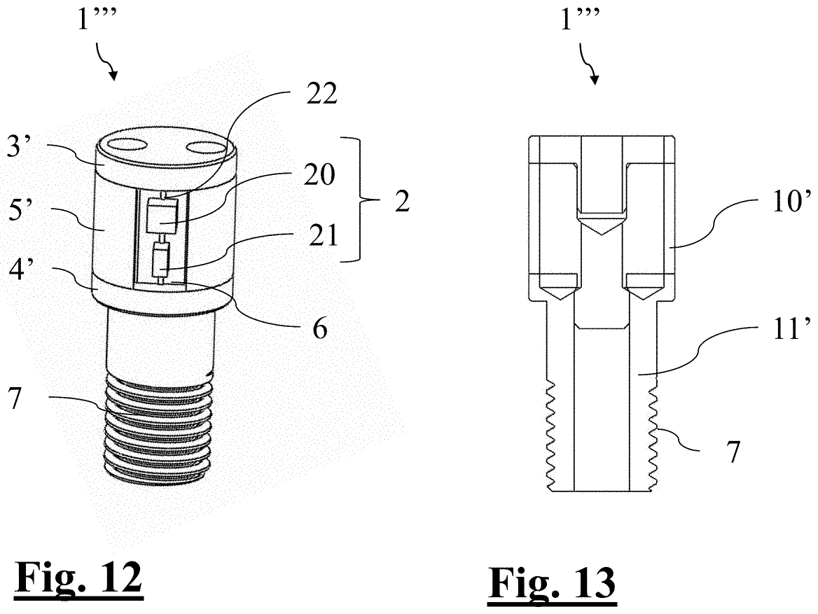

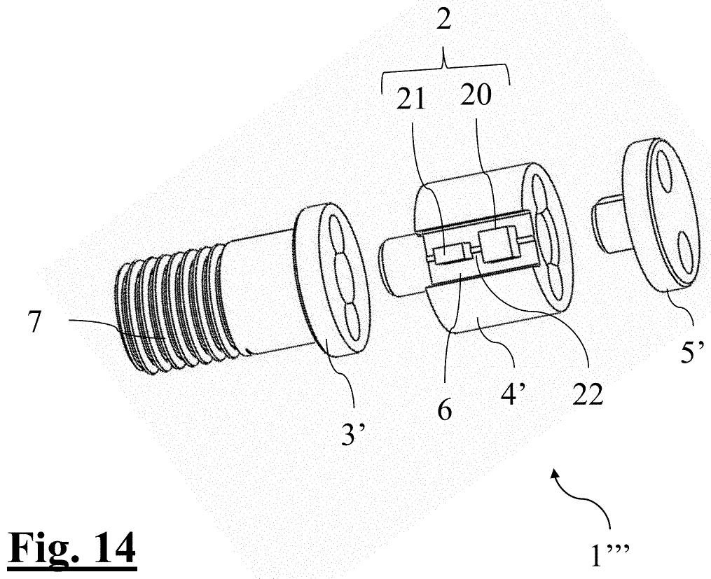

With reference to the enclosed FIGS. 12-14, reference number 1′″ identifies a fourth embodiment of a sensored component according to the present invention.

The fourth embodiment is substantially identical to the second one; however, it has a smaller diameter and as a result it is arranged to couple to one of the endosseous screws 101 of smaller diameter of the bone screw system 400.

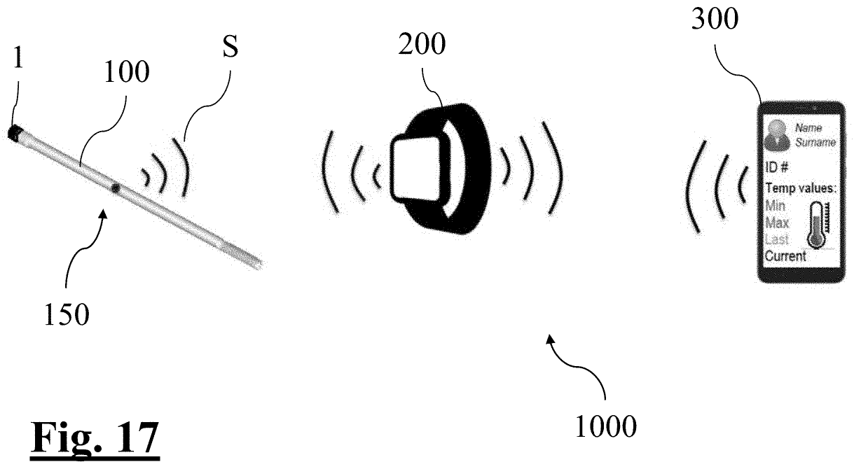

With reference to the enclosed FIG. 17, reference number 1000 identifies a monitoring system according to the present invention.

On the one side, the system comprises a sensored screw assembly 150, made of an endosseous screw 100 provided with the previously described sensored component 1.

The system still includes an RFID 200 reader, provided with a wrist strap or otherwise wearable by the user. The RFID reader 200 is arranged to query the transponder RFID of the sensored screw 150, in particular obtaining the following data:
a unique identification of the user;
data relating to the endosseous screw (model, size, implantation date and position);
a degree of strength of the coupling between the receiving reader and the endosseous screw;
local temperature T and/or pH P, acquired in real time.

Moreover, the RFID reader 200 is able to connect, for instance through Bluetooth technology, to a mobile device 300 on which a proper application for viewing the above data received from the reader is installed.

Moreover, the receiving reader may store data and send alarms should warning thresholds for temperature T and pH P be exceeded.

An advantage of the invention lies in the fact that the sensored implant is applied to normal endosseous screws. Therefore, the same screw may be implanted with or without sensored component, depending on the specific needs and operative choices.

A further advantage derives from the extreme simplicity of the assembling procedure of the sensored component by screwing it onto the screw head. The presence of a coupling element for a clamping tool further favors said assembly, which may be performed by the surgeon even in the intra-operative phase.

Another advantage of the present invention is the use of the stem of the endosseous screw as a radiating antenna by the sensored component. In this way, a good signal communication to the external reader is achieved without resorting to a redesign of the system.

Still another advantage lies in the fact that the sensored component is provided with a suitable adaptation circuit that easily allows tuning the device to the different UGF RFID working frequencies and to the different morphologies of the patient.

Still another advantage derives from the use of an RFID transponder, which allows the signal transmission even without using internal batteries which would limit the useful life of the device.

Obviously, a person skilled in the art, in order to satisfy contingent and specific requirements, may make numerous modifications and variations to the invention, all of them by the way included in the scope of protection of the invention as defined by the following claims.

What is claimed is:

1. A component for endosseous screw, provided with a main body which supports electronics comprising at least one signal emission circuit and at least one sensor for acquiring one or more biophysical parameters of a patient, said component being associable with an endosseous screw, said signal emission circuit being arranged to send at least one signal relating to the one or more biophysical parameters acquired by the at least one sensor.

2. The component according to claim 1, wherein said signal emission circuit is an RFID transponder circuit.

3. The component according to claim 1, wherein the main body comprises a distal portion and a proximal portion which are both conductive separated by a dielectric intermediate portion, said electronics being electrically connected to both said distal portion and said proximal portion.

4. The component according to claim 3, wherein said electronics comprises at least one integrated circuit arranged on a conductive bridge which connects said distal portion to said proximal portion.

5. The component according to claim 4, wherein the signal emission circuit and the at least one sensor are formed on said integrated circuit.

6. The component according to claim 4, wherein said integrated circuit and said conductive bridge are housed in a side recess of said intermediate portion.

7. The component according to claim 3, wherein said main body is monolithic and made of a dielectric material, the distal and proximal portions being made conductive by a metallization or metal deposition process on the surface of said dielectric material.

8. The component according to claim 3, wherein said main body is composed by said distal and proximal portions of a conductive material assembled at the two opposite ends of the intermediate portion of a dielectric material.

9. The component according to claim 1, comprising an attachment portion for coupling with an endosseous screw, said attachment portion extending in a proximal position with respect to the main body.

10. The component according to claim 9, wherein said attachment portion comprises a cylindrical stem provided with an outer thread arranged to couple with the endosseous screw.

11. The component according to claim 1, wherein the one or more biophysical parameters acquired by the at least one sensor comprises at least one local temperature and/or pH of the patient, and they represent an indicator of possible infections at the implanted endosseous screw.

12. A screw assembly comprising an endosseous screw comprising a tip, a stem and a head and at least one component according to claim 1 coupled to said head.

13. A screw assembly according to claim 12, wherein said endosseous screw is at least partially made of a conductive material and it defines, together with the component, an antenna for the signal emission circuit.

14. A screw assembly according to claim 12, wherein said endosseous screw is arranged to treat bone collapses or deformations, said tip and said head being both threaded, said stem being unthreaded.

15. A monitoring system comprising a screw assembly according to claim 12 and at least one receiving reader arranged to acquire a signal coming from the signal emission circuit defined by the screw assembly, said signal comprising at least data relating to the one or more biophysical parameters acquired by the at least one sensor.
